(19) United States
(12) Patent Application Publication
(10) Pub. No.: US 2020/0261496 A1
(43) Pub. Date: Aug. 20, 2020

(54) Method of treating and/ or preventing viral infection.

(71) Applicants: Vladimir Oks, Medford, MA (US); Andrey Oks, Freehold, NJ (US)

(72) Inventors: Vladimir Oks, Medford, MA (US); Andrey Oks, Freehold, NJ (US)

(21) Appl. No.: 16/825,441

(22) Filed: May 7, 2020

(51) Int. Cl. A61K 33/00; A61P 31/14

(52) U.S. Cl. CPC A61K 33/00; A61P 31/14

(57) ABSTRACT

A method of treating and/or preventing viral infections including common cold, influenza, and coronaviruses with or without symptoms is based on a fact that virus's survival time depends on the temperature and relative humidity, and an assumption that a virus can be killed or its ability to penetrate and replicate copies inside living cells can be weakened, by prolonged exposure to such environment, which can be controlled by an individual. Creating such environment in the upper respiratory and gastrointestinal (GI) tracts and preventing spread of the virus into the lower part of the respiratory tract using continuous and prolonged heat exposure on the virus, prevents development of pneumonia.

### FIELD OF THE INVENTION

The present invention relates to the treatment of a viral infection by heat therapy. The objective of the present invention is to destroy viruses by heat in the upper respiratory and gastrointestinal (GI) tracts and prevent the development of pneumonia and other complications. Study of this method is based on a human body temperature regulation model, represented by a feedback control system under the influence of viruses and the effect of the present method.

### BACKGROUND OF THE INVENTION

Cold

The common cold is a very frequent acute illness in industrialized societies and the leading cause of visits to the physicians in the United States. Though it is usually benign, it is a leading cause of absence from work, also causing a significant economic burden including loss of productivity and treatment costs. The common cold is caused by a variety of viruses, most of the time of the rhinovirus and corona types. There is currently no cure for common cold, so that current therapy targets symptoms relief. Prevention strategies for the common cold include avoiding infected people and frequent hand washing during cold season.

Conventional therapies have a limited efficacy. Certain drugs are costly and have side effects. Dietary supplements (e.g., Echinacea, ginseng, etc.) are often believed to be effective, but they lack formal studies using modern culture methods.

Flu

Flu is a contagious illness caused by the influenza virus. Common flu is characterized by an acute infection associated with respiratory problems, intense muscle pain, headaches, chills, nasal obstruction, fever, cough, sneezing and sore throat. The majority of the population will generally recover from flu without any complication. Older people, young children and people with deficient immune systems, may have serious complications or may even die from the flu.

Current literature describes human common influenza as a serious disease causing each year an estimated 36,000 deaths in the United States only. Yearly common influenza (flu) epidemics result in lost workdays and schooldays as well as a significant number of hospitalization days and even deaths among the elderly, seniors being particularly vulnerable to respiratory infections.

New influenza strains infecting humans therefore appear from time to time. These strains frequently originate from other species and have adapted to human through mutations. The swine influenza A H1N1 flu virus, also named Swine Flu, has been reported around the world and was declared a pandemic influenza virus. Swine flu H1N1 originally only affected pigs but started infecting humans in North America in 2009. Humans having little to no natural immunity to this virus, it can lead to a serious and widespread illness.

Vaccines can prevent flu in 70 to 90% of the cases observed in healthy adults. However, the flu vaccine has to be redesigned each year because the previous years vaccines are likely to be ineffective against the newly mutated strains. The yearly vaccine is therefore designed before the season begins based on that from the previous year. Since scientists usually cannot predict with accuracy which strain will be dominant, the vaccine may not fully correspond to the new strain. In addition, the flu vaccine is specifically designed against influenza virus strains A and B and does not therefore protect against cold viruses such as rhinovirus and corona types.

Flu symptoms can be alleviated with either the previously mentioned treatment or with, for example, 1) over-the-counter analgesics to relieve pain and reduce fever; 2) cough suppressants for dry cough with no mucus; 3) expectorants to help clear mucus so it can be coughed up; and/or 4) decongestants to reduce nasal congestion.

Coronaviruses

Coronaviruses vary significantly in risk factor. Some can kill more than 30% of those infected (such as MERS-CoV), and some are relatively harmless, such as the common cold. Coronaviruses cause colds with major symptoms, such as fever, and sore throat from swollen adenoids, occurring primarily in the winter and early spring seasons. Coronaviruses can cause pneumonia (either direct viral pneumonia or a secondary bacterial pneumonia) and bronchitis (either direct viral bronchitis or a secondary bacterial bronchitis). The much publicized human coronavirus discovered in 2003, SARS-CoV, which causes severe acute respiratory syndrome (SARS), has a unique pathogenesis because it causes both upper and lower respiratory tract infections.

Many scientific publications provide data on the dependence of virus survival time on temperature and humidity on surfaces of different materials.

For example, coronavirus Survival Times at Relative Humidity & Air Temperatures in Hours & Days: (K. H. Chan, 2011; Smith RD, 2006)
The survival times at 80% RH and 40° C. [104° F.] were less than 7 hours for proxies of coronaviruses on stainless steel.*
The survival times at 50% RH and 40° C. [104° F.] were more than 24 hours.* There was a reduction in the ratio of virus to −3 Log 10 (Nt/N0) in 24 hours.
The survival times at 20% RH and 40° C. [104° F.] were more than 120 hours.* There was a reduction in the ratio of virus to −3 Log 10 (Nt/N0) in 120 hours.
The survival times at 50% RH and 20° C. [68° F.] were less than 7 days on stainless steel.*
* For proxies of coronaviruses on stainless steel. The proxies used were transmissible gastroenteritis virus (TGEV) and mouse hepatitis virus (MHV).

Different scientific papers have different data, but the coronavirus's survival time tendency to depend on temperature and relative humidity continues.

Viruses spread from person to person primarily via respiratory droplets from coughing or sneezing. People are thought to be most contagious when symptomatic, though some spread might be possible before symptoms show. The time between exposure and symptom onset is typically five days, but may range from two to fourteen days. Common symptoms include fever, dry cough and shortness of breath. Complications may include pneumonia and acute respiratory distress syndrome. There is no vaccine or specific antiviral treatment, but research is ongoing. Efforts are aimed at managing symptoms and supportive therapy. Recommended preventive measures include handwashing, covering the mouth when coughing, maintaining distance from other people (particularly those who are unwell), and monitoring and self-isolation for fourteen days for people who suspect they are infected.

Public health responses have included national pandemic preparedness and response plans, travel restrictions, quarantines, curfews, event postponements and cancellations, and facility closures. These include a quarantine of Hubei, China; the nationwide quarantines of Italy, Spain, the Czech Republic, and Germany; curfew measures in China and South Korea; various border closures or incoming passenger restrictions screening methods at airports and train stations; and travel advisories regarding regions with community spread.

Symptoms of COVID-19 are non-specific and those infected may either be asymptomatic or develop flu like symptoms such as fever, cough, fatigue, shortness of breath, or muscle pain.

Further development can lead to severe pneumonia, acute respiratory distress syndrome, sepsis, septic shock and death. Some of those infected may be asymptomatic, returning test results that confirm infection but show no clinical symptoms, so researchers have issued advice that those with close contact to confirmed infected people should be closely monitored and examined to rule out infection.

### HISTORICAL SUMMARY OF THE INVENTION

This method is based on the theory of anthropogenesis sciences, studying the process of historical and evolutionary formation of humans. The method is a natural extension of human evolutionary development, since the human use of fire, in the fight for its existence, in the fight against foreign bodies in an organism including in the fight against viruses.

This method is subject to the science of biology, the natural science that studies life and living organisms especially its immunology branch that covers the study of immune systems in all organisms by the physiological functioning of the immune system in both state of health and state of illness, and the body's response to foreign bodies. The main purpose of the immune system is to protect against intrusions and to preserve the integrity of the internal environment of the body, its biological individuality.

This method is subject to the laws of cybernetics, the science of general patterns of information management and transmission processes in machines, living organisms and society. Cybernetics includes the study and impact of feedback, on management and communication in living organisms, machines and organizations, including self-organization, and regulation of all processes in nature including human activity and society.

This method is subject to the laws of mechanics—a section of physics, science that studies the movement of material bodies and the interaction between them. An individual is the most complex object not only on Earth, but perhaps in the entire universe. Taking into account the evolutionary development of man and progress of modern technologies, man can be considered as a material, mechanical object, at least its physical body's parts and systems describe by physics, physiology, biology and microbiology using simplified description of any human function by a small number of mathematical equations.

This method is subject to the laws of energy—a section of physics, science that studies the quantitative property that must be transferred to an object in order to perform work on, or to heat, the object. Energy is a conserved quantity; the law of conservation of energy states that energy can be converted in form, but not created or destroyed. Common forms of energy include the chemical energy released when a fuel burns and the thermal energy due to an object's temperature.

This method is subject to the science of chemistry—the discipline, which is involved with the structure, properties and behavior of elements and compounds and the changes they undergo during a reaction with other substances, specifically its sub discipline of biochemistry's field of metabolism, the set of life-sustaining chemical reactions in organisms.

This method is subject to the laws of the continuous-time world. Its main property is the continuity of the time variable, i.e. there is a finite value of the any process's function in any moment of the time. The functioning of all phenomena in nature, including human nature, is carried out in an integral dependence on changes under the influence of internal and external impacts.

### SUMMARY OF THE INVENTION

A method of treating and/or preventing viral infections including common cold, influenza, and coronaviruses with or without symptoms is based on a fact that virus's survival time depends on the temperature and relative humidity, and an assumption that a virus can be killed or its ability to penetrate and replicate copies inside living cells can be weakened, by prolonged exposure to such environment, which can be controlled by an individual. Creating such environment in the upper respiratory and GI tracts and preventing spread of the virus into the lower part of the respiratory tract using continuous and prolonged heat exposure, prevents development of pneumonia.

The heat can be delivered to the respiratory tract by different means. With the present method, an agent delivering heat is pure, hot, boiled water the simplest, cheapest and natural product for a human. The method further reduces the risk of pneumonia by washing viruses away from the respiratory tract to GI tract. At the same time the hot water prevents development of a viral infection in the GI and urinary tracts.

The present method prevents or reduces the spread of a virus into the lower respiratory tract and prevents development of pneumonia, most effectively at the early stages of an illness, when an infection just starts to develop in the upper respiratory and GI tracts. A virus can be killed or its ability to penetrate and replicate copies inside living cells can be weakened, using continuous and prolonged hot water exposure on the virus.

It is an object of the present invention to create an environment in the upper respiratory and GI tracts to prevent spread of the virus into the lower part of the respiratory tract and prevent development of pneumonia.

It is an object of the present invention to provide easily administered preparations that can be used at home by millions of people without medication or special equipment.

It is another object of the present invention to use this method as a preventive measure. It is an additional proactive preventive measure, similar to a recommendation of “washing your hands”.

A further object of the present invention is to provide a safe method with no side effects.

It is additional object of the present invention to use pure, hot, boiled water the simplest, cheapest and natural product for a human, as an agent delivering heat.

A still further object of the present invention is to provide preparations that are easy to administer and can be carried out and used anywhere so the user can continue the therapies throughout the day, days, weeks, and months.

It is also an objective of the present invention to treat and prevent viral infection and to help kill other foreign bodies inside the GI and urinary tracts.

It is another objective of this invention to open a new chapter in research of hyperthermia application of this method for treatment other foreign bodies inside human organism including different type of cancer cells and creation of special medical devices for treatment and prevention procedures.

### BRIEF DESCRIPTION OF THE DRAWING

This and/or other aspects and advantages of the present invention will become apparent from the following description of preferred embodiment, taken in conjunction with the appended drawings, in which:

FIG. 1 illustrates simplified model of a human body temperature regulation system, similar to a block diagram of feedback control system with a conventional integral controller.

FIG. 2 illustrates simplified model of a human body temperature regulation system under influence of a virus as a foreign body, introduced into a human organism, similar to a block diagram of feedback control system with a conventional integral controller and external source of disturbance.

FIG. 3 illustrates simplified model of a human body temperature regulation system under influence of a virus as a foreign body, into a human organism and usage of the present invention, similar to a block diagram of feedback control system with a conventional integral controller, external source of disturbance and disturbance compensator.

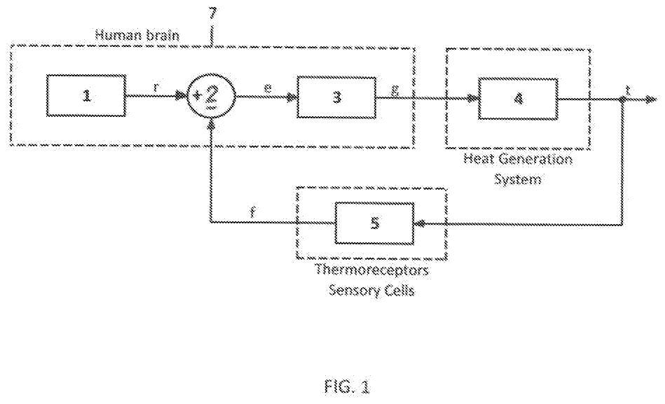

In FIG. 1 a set point unit 1 generates an input reference signal r, which is compared with the feedback signal f at the negative input of the summing unit 2, which in this case computes the difference between the signals, to obtain the deviation signal e. The deviation signal e is inputted into the integral controller 3 having transfer function Ki/s. The controller 3 generates, correspondingly, the integral variable signal g. The signal g is inputted into the controlled system block 4. The block 4 generates the output variable function t that is inputted into the feedback sensor block 5. The feedback sensor output signal f is entered into the negative input of the summing unit 2.

It is known that the transfer function of the integral controller is Gc(s)=Ki/s Where:

s—a Laplace operator;

Ki—a gain of integral controller 3 and Ki=1/Ti;

Ti—a time constant of integration of integral controller 3.

FIG. 1 is also illustrated by a simplified model of a human body temperature regulation system, as described below.

The Human Brain block 7 is an organ that serves as the center of the nervous system. Brain exerts centralized control over a body's nervous system and other organs by generating patterns for body activity. This centralized control allows rapid and coordinated responses to changes of internal and external conditions. Sophisticated, purposeful control of behavior based on a complex sensory input requires information integrating capabilities of a centralized brain.

The Human Brain block 7 includes a set point unit 1, the comparison device 2 and the regulator 3 that are parts of the regulation center of human body temperature—the Hypothalamus. Block 5—regular Thermoreceptors Sensory Cells, where free nerve endings that reside mostly in the skin, liver, and skeletal muscles transduce sensory feedback information f to the central nervous system in the brain block 2 in the hypothalamus. This feedback signal f is entered into the Brain block 2's input, with the opposite sign to the reference signal r, as negative feedback, to stabilize set body temperature t. The computed difference e between the signals is inputted into the integral regulator 3, which is also a part of the central nervous system Human Brain block 7.

The regulator's output signal g changes gradually and continuously. The output signal g change rate is determined by a parameter called the time constant of integration Ti and for each person is its own internal constant of temperature regulation. This signal g is inputted into the Heat Generation System of man's body, block 4 that maintains or changes body temperature. In thermoregulation of warm-blooded organisms, body heat is generated mostly in the deep organs, especially the liver, heart, in contraction of skeletal muscles, by vasoconstriction, shivering, sweating, hormones, metabolism, endothermy, and homeothermy mechanisms of the temperature regulation of human body.

For example, the set point block 1 of the Human Brain 7 instructs to set body temperature at 36.7° C. (98.1° F.). The feedback sensor, Thermoreceptors Sensory Cells block 5 that measure temperature t of the body generates feedback signal f that is compared in the device 2, of the Human Brain's block 7 with an opposite sign to the reference signal r. Because signal f equal to signal r, the deviation signal e of the block 2 equal to zero. There is no signal into Heat Generation System of man's body block 4. The Heat Generation System maintain body temperature, that corresponds to the specified by set point of Human Brain 36.7° C. (98.1° F.). In reality, the temperature cannot be kept perfectly at 36.7° C. (98.1° F.), as the process is integral, constantly and continuously changing, in the form of a saw, within man's body sensitivity, for example between 36.71° C. and 36.69° C.

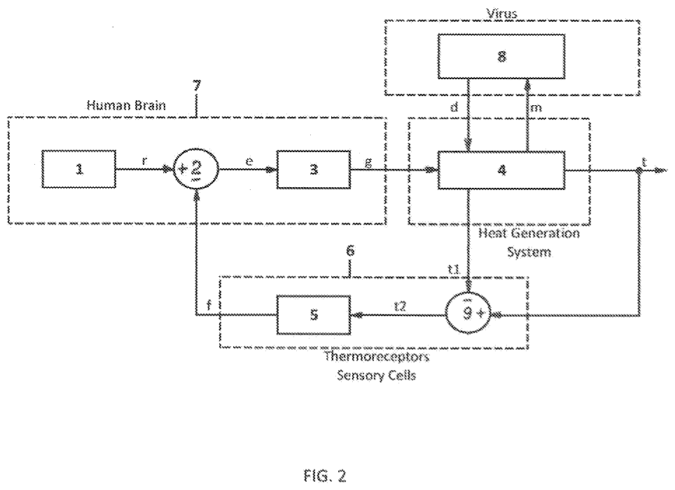

FIG. 2 is illustrated by FIG. 1 and presence of foreign bodies—viruses introduced into a human organism, to produce the same effect as an external source of disturbance into a block diagram of feedback control system with a conventional integral controller.

The external source of disturbance block 8 generates a disturbance signal d that is inputted into the controlled system block 4.

In this model of human body temperature regulation system under influence of a virus as a foreign body, Virus is a source of disturbance block 8 and directly effects Heat Generation System block 4 by signal d. Concurrently, Heat Generation System block 4 directly responds to the Virus, the source of disturbance block 8, by signal m. The Heat Generation System block 4 under influence of viruses generates temperature disturbance signal t1 that inputs into the summing unit 9—disturbance part of the Thermoreceptors Sensory Cells block 6. The output signal t2 of the summing unit 9 inputs into block 5, the regular part of the Thermoreceptors Sensory Cells block 6. This signal t2 is a combined signal of the temperature signal t from the positive input and the temperature disturbance signal t1 from the negative input of the summing unit 9.

At the beginning, the Virus block 8 disturbs body temperature balance by signal d, disrupting operations of certain mechanisms of the system generating heat in the human body and creating a favorable environment for itself. On the other hand, the Heat Generation System, block 4 converts, according to the law of energy conservation, and transfers part of the heat energy to counteract and fight viruses by signal m, influencing Virus block 8. The Heat Generation System under the influence of viruses generates temperature disturbance signal t1. Within block 6, the summing unit 9 computes the difference between signal t from the positive input and signal t1 from the negative input, generating output signal t2 entering block 5, regular part of the Thermoreceptors Sensory Cells.

Under the influence of the Virus's disturbance source, the summing temperature signal t2 is decreased. The feedback signal f of the Thermoreceptors Sensory Cells block 6 is decreased. The deviation signal e of block 2 is increased because reference signal r from the positive input is greater than feedback signal f from the negative input. The Human Brain regulator block 3, output signal g is changed gradually and continuously. The output signal g change rate, determined by the time constant of integration, depends only on the difference of signals r and f. This signal g is inputted into Heat Generation System of man's body block 4 that increases output t of man's body temperature according to the integration signal g of the Human Brain regulator block 3 gradually and continually. The temperature t is increased until the feedback signal f is equal to the reference signal r and deviation signal e is equal to zero. The body temperature t comes to a new equilibrium state, for example 37.4° C. (99.3° F.).

Similarly, the human immune system responds to foreign organisms by increasing body's temperature to create an environment that helps kill viruses or weaken virus's ability to penetrate and replicate inside living cells.

In a simplified representation of the disturbance source Virus block 8, a more severe virus infection in the upper respiratory and GI tracts, more significantly disturbs the temperature balance. Disturbance temperature signal t1 is increased more. The output signal t2 of the summing unit block 9 is decreased accordingly. The feedback signal f of the Thermoreceptors Sensory Cells, block 6 is decreased. In turn, deviation signal e is increased and Human Brain regulator block 3, output signal g is changed gradually and continuously, but more rapidly due to a greater difference between signals r and f. The temperature t is increased more rapidly until the feedback signal f is equal to the reference signal r and deviation signal e is equal to zero. The body temperature t comes to a new equilibrium state, for example at 39.5° C. (103.1° F.).

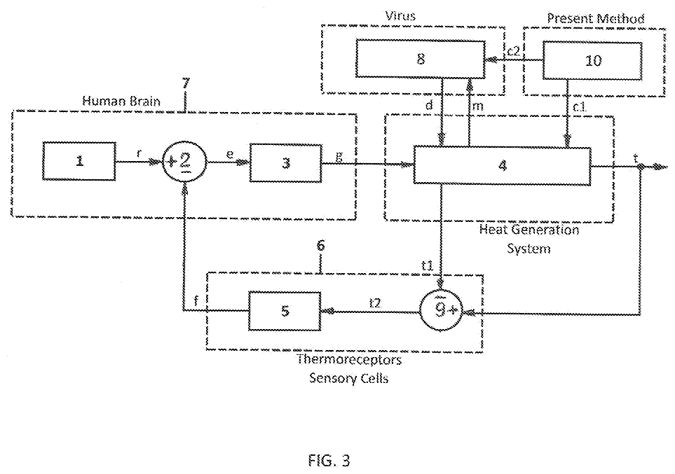

FIG. 3 is illustrated by FIG. 2 and usage of the present invention as a disturbance compensator into block diagram of feedback control system with a conventional integral controller and external source of disturbance.

### DETAILED DESCRIPTION OF THE INVENTION

A method of treating and/or preventing viral infections including common cold, influenza, and coronaviruses with or without symptoms is based on a fact that virus's survival time depends on the temperature and relative humidity, and an assumption that a virus can be killed or its ability to penetrate and replicate copies inside living cells can be weakened, by prolonged exposure to such environment, which can be controlled by an individual. Creating such environment in the upper respiratory and GI tracts and preventing spread of the virus into the lower part of the respiratory tract using continuous and prolonged heat exposure on the virus, prevents development of pneumonia.

An agent delivering heat is pure, hot, boiled water the simplest, cheapest and natural product for a human. The method further reduces the risk of pneumonia by washing viruses away from the respiratory tract to GI tract. At the same time the hot water prevents development of a viral infection in the GI and urinary tracts.

The present method prevents or reduces the spread of a virus into the lower respiratory tract and prevents development of pneumonia, most effectively at the early stages of an illness, when an infection just starts to develop in the upper respiratory and GI tracts, when first signs of symptoms appear, or when an individual has been tested positive for a virus, but is asymptomatic. The virus can be killed or its ability to penetrate and replicate copies inside living cells can be weakened, using continuous and prolonged hot water exposure on the virus.

For a better understanding of the present invention, reference is made to the accompanying FIG. 3, which illustrates a simplified model of a human body temperature regulation system under influence of a virus as a foreign body and usage of the present invention, similar to a block diagram of feedback control system with a conventional integral controller, external source of disturbance and disturbance compensator.

The disturbance compensator block 10 generates a disturbance compensator signal c1 that is inputted into controlled system block 4 and disturbance compensator signal c2 that is inputted directly into external source of disturbance block 8.

In this model of human body temperature regulation system under influence of a virus as a foreign body, the disturbance compensator block 10 represents Present Method of treating and preventing viral infection.

The temperature t of man's body is increased by continuous drinking of pure hot, boiled water with a consumption rate of 0.5 liters or 0.13 gallons per 10 to 12 minutes, with water temperature range of 55° C. to 75° C. or 131° F. to 167° F. Temperature t is increased for two reasons:

1. The temperature is increased especially in the upper respiratory and GI tracts, in the place of infection because the water is hot and adds heat energy, improves metabolic process and blood circulation. The Present Method represented by the Disturbance Compensator block 10 generates output signal c1. This signal c1 compensates signal d by entering directly into the Heat Generation System block 4.

2. The continuous and prolonged drinking of hot water reduces the number and virulence of the viruses. The Present Method represented by the disturbance compensator block 10, generates output signal c2. This signal c2 unloads signal m by adding heat energy from the outside and reduces temperature disturbance signal t1. Hot water is consumed with the duration in the range of 2 to 3 hours and the amount of 5 to 9 liters or 1.3 to 2.4 gallons. The objective of the present method is to kill as many viruses as possible and/or weaken its ability to penetrate and replicate copies inside living cells and to help the immune system.

The number of viruses that disturb temperature balance of the human body is reduced. The body temperature t is increased and temperature's disturbance signal t1 is decreased. The summing temperature signal t2 is increased. The feedback signal f of the Thermoreceptors Sensory Cells is increased accordingly. The deviation signal e of the block 2 is decreased because feedback signal f from the negative input is increased. The regulator's output signal g of the Human Brain block 3 is decreased gradually and continuously with the change rate determined by the time constant of integration, and is dependent only on the difference of signals r and f. This signal g is inputted into the Heat Generation System of man's body block 4 that decreases man's body temperature output t. The Present Method's, disturbance compensator block 10 helps the immune system to counteract the influence of the Virus block 8 by killing viruses and/or weakening its ability to penetrate and replicate copies inside living cell of organism. Immune system consumes a lot of energy to fight viruses. Present Method's block 10 unloads the immune system in the fight against the Virus block 8 and takes some of the energy consumption on itself.

The temperature t is decreased until the feedback signal f is equal to the reference signal r and deviation signal e is equal to zero. The body temperature t comes to a new equilibrium state, for example back to 37.4° C. (99.3° F.) from 39.5° C. (103.1° F.).

The longer hot water is consumed the more viruses can be killed or weakened, the greater the possibility that body temperature t returns to normal and human body temperature regulation system returns back to the conventional block diagram described by FIG. 1. The body temperature t is a good indicator of the severity of the disease and the correctness of the application of the present method.

### PRACTICAL USAGE OF THE INVENTION

Treatment

This method is the most effective for treatment during the early stages of an infection, when first signs of symptoms appear or when an individual has been tested positive for a virus, but is asymptomatic.

If individual experiences symptoms such as fever, sore throat, cough, shortness of breath, muscle pain and so on, he/she should begin following the presented method immediately.

Treatment Procedure:
1. Boil regular water and pour into a cup/mug and allow cooling to the point where it can be safely consumed, based on an individual's ability.
2. Start drinking the hot water at the maximum temperature that an individual can comfortably drink. Water temperature range for an average adult individual should be: 55° C. to 75° C. or 131° F. to 167° F.
3. Water should be consumed at a constant, steady pace.
4. Once the first cup is finished, continue steps 1 through 3, with minimal breaks between cups, to ensure a continuous steady pace of consumption.

Continue with steps 1 through 4, either for:
a. For 2 to 3 hours. The average amount of water consumed for an average adult is 5 to 9 liters or 1.3 to 2.4 gallons depending on the virus type, the stage of the illness, and individual; or consumption rate of 0.5 liters or 0.13 gallons per 10 to 12 minutes.
b. Symptoms experienced prior to applying this method have gone away or have been significantly reduced. Light cough may persist for a little while longer.

An individual should expect some or significant amount of sweating throughout the process and more frequent urination.

Prevention

Prevention method should be utilized in a similar manner as other proactive steps identified by medical professionals, such as washing your hands, using hand sanitizer or wearing a facemask, if it is necessary. This method could be used more frequently during virus seasons and any time when an individual feels they were in a contaminated environment (e.g. around sick people). It is a good practice to follow on a consistent basis.

Prevention Procedure:

Repeat with steps 1 through 4 of the above described Treatment Procedure, but with the duration of hot water exposure of 0.5 to 1 hours. The average amount of water consumed for an average adult is 1.3 to 2.5 liters or 0.33 to 0.66 gallons, or consumption rate of 0.5 liter or 0.13 Gallon per 10 to 12 minutes.

Depending on an individual's internal condition, overall medical and health condition, as well as external environment, the treatment and/or prevention procedures can be used in different combinations, with different frequency, within a day, days, weeks or months. This method does not treat bronchitis or pneumonia. It is the most effective at the early stage of illness. It can expedite recovery and prevent complications.

The present invention can be used as a stand-alone method of treatment and prevention viral infection as well as additional to all existing conventional methods.

This method is safe, and there are no side effects. However, people with special health conditions have to use this method with permission of their physician or under physician's control. It is a personal responsibility of each individual to use this method appropriately and based on the user's physical and health condition, without causing harm to self.

Time frame of usage of the present method.

### Example 1

First day:
1. 11.00 AM. An individual takes their temperature for the first time because he/she feels sick. Temperature is 39.5° C. (103.1° F.).
2. 11.15 AM. Individual starts to drink hot boiled water according to the Treatment Procedure described above.
3. 3.00 PM. Individual takes their temperature again. If temperature is significantly less than 39.5° C. (103.1° F.), for example 37.4° C. (99.3° F.) no further actions is required pertaining to the method on this day. If temperature is more than 38° C. (100.4° F.) the individual should use the Prevention Procedure described above 4.00 PM-5.00 PM on the same day.

Second day:
4. If by 12 PM temperature has not increased, treatment by this method is complete.
5. If by 12 PM temperature has increased significantly, repeat the Treatment Procedure again, one more time.

When the Treatment Procedure is finished the usage of this method is done. Furthermore, in all circumstances, the conventional methods of treatment, including medical assistance, should be considered and followed appropriately.

### Example 2

First day:
1. 5.00 PM. An individual takes their temperature for the first time because he/she feels sick. Temperature is 40.5° C. (105° F.).
2. 5.15 PM. Individual starts to drink hot, boiled water according to the Treatment Procedure described above.
3. 9.00 PM. Individual takes their temperature again. If temperature is significantly less than 40.5° C. (105° F.), for example 37.4° C. (99.3° F.) no further action is required pertaining to the method, on this day. If temperature is more than 38° C. (100.4° F.), the individual may take fever reducer or multi symptoms relief medicine and go to bed.

Second day:
4. If in the morning, for example 8.00 AM, the temperature is still high. Individual should start to drink hot, boiled water according to Treatment Procedure described above.
5. 12.00 PM. Individual takes their temperature again. If temperature has dropped significantly, no further action is required pertaining to the method, on this day. If temperature is more than 38° C. (100.4° F.) the individual should use the Prevention Procedure described above 1.00 PM-2.00 PM on this day.
6. 4.00 PM. Individual takes their temperature again. If temperature is significantly less, for example 37.4° C. (99.3° F.), treatment by this method is complete.
If temperature is still high, after using the present method for two days, as presented in the examples above, the conventional methods of treatment, including medical assistance, should be considered and followed appropriately.

Testing

### Example 1

This method was tested late December 2012 by the author of this invention for treatment of flu. During the onset of slight symptoms of sore throat and muscle pain in the legs, the individual started to follow this method's Treatment Procedure immediately as described above. The individual achieved full recovery, with none of the original symptoms present, within one day.

### Example 2

This method was tested late February 2020 by the author of this invention for treating presumably coronavirus COVID-19. During the onset of symptom of high fever 40.5° C. (105° F.), the individual started to follow this method's Treatment Procedure immediately as described above. The individual's fever was gone within one day. The individual was isolated for the next 6 days to prevent infecting other people. The individual continued to experience residual light cough, lasting 4 weeks. After the first two weeks of the cough, the individual visited a doctor and took a chest X-rays to confirm that the lungs were clear and that the infection did not develop into pneumonia.

Every individual who has used this method has recovered expeditiously and without any complications.

What is claimed:

1. A method of treating and/or preventing viral infections including common cold, influenza, and coronaviruses with or without symptoms, based on continuous drinking of pure hot, boiled water with consumption rate of 0.5 liters or 0.13 gallons per 10 to 12 minutes, at the maximum temperature that a person can comfortably drink, with a range for an average adult individual of 55° C. to 75° C. or 131° F. to 167° F.

2. The method of claim 1, wherein for treating a viral infection the duration of hot water exposure on a virus is 2 to 3 hours with the average amount of water consumed for an average adult is 5 to 9 liters or 1.3 to 2.4 gallons, depending on the virus type, the stage of the illness, and individual; or until an individual feels the symptoms have disappeared or have been significantly reduced.

3. The method of claim 1, wherein for preventing a viral infection the duration of hot water exposure is 0.5 to 1 hours with the average amount of water consumed for an average adult is 1.3 to 2.5 liters or 0.33 to 0.66 gallons.
